(19) United States
(12) Patent Application Publication
(10) Pub. No.: US 2026/0327255 A1
(43) Pub. Date: Oct. 22, 2026

(54) LIGAND-DIRECTED COVALENT MODIFICATION OF AMINO ACID SIDE CHAINS USING CYCLIC IMINE MANNICH ELECTROPHILES

(71) Applicant: Purdue Research Foundation, West Lafayette, IN (US)

(72) Inventors: Casey John Krusemark, West Lafayette, IN (US); Emily C. Dykhuizen, West Lafayette, IN (US); Sijie Wang, Mountain View, CA (US)

(21) Appl. No.: 19/119,347

(22) PCT Filed: Oct. 11, 2023
(86) PCT No.: PCT/US2023/034916

§ 371 Date: Apr. 8, 2025

Related U.S. Application Data
(60) Provisional application No. 63/416,142, filed on Oct. 14, 2022.

(51) Int. Cl. C07K 1/107

(52) U.S. Cl. CPC C07K 1/1077

(57) ABSTRACT

A method of covalently modifying tyrosines, tryptophans, and pyrrolated lysines in a protein comprising reacting the side chains of the amino acids with a cyclic imine Mannich electrophile appended to a ligand that binds to the protein; and a protein comprising a ligand binding site, which comprises one or more tyrosine, tryptophan or pyrrolated lysine residues, the side chains of which have been selectively covalently modified with a cyclic imine.

### CROSS REFERENCE TO RELATED APPLICATIONS

This present patent application relates to and claims the priority benefit of U.S. Provisional Application Ser. No. 63/416,142, filed Oct. 14, 2022, the content of which is hereby incorporated by reference in its entirety into this instant disclosure.

### GOVERNMENT SUPPORT

This invention was made with government support under R35GM128894 awarded by the National Institutes of Health. The US government has certain rights in the invention.

### TECHNICAL FIELD

The present disclosure relates to methods of ligand-directed, covalent modification of tyrosines, tryptophans, and pyrrolated lysines in proteins using cyclic imine Mannich electrophiles.

### BACKGROUND

The number and diversity of covalent inhibitors has increased dramatically in recent years. The majority are targeted covalent inhibitors (TCIs), which are rationally designed to bind to a protein target and then form a covalent bond. A TCI brings a reactive moiety (typically an electrophile of low reactivity) directly adjacent to an amino acid side chain, inducing a high effective concentration. This leads to a selective covalent conjugation to a proximal amino acid side chain to yield an inhibitor with irreversible occupancy (Gehringer et al. J. Med. Chem. 2019, 62(12), 5673-5724; Baillie et al. Angew. Chemie. Int. Ed. 2016, 55(43), 13408-13421.

Covalent labeling has multiple advantages as a tool in probe and drug development (Ghosh et al. Chem Med Chem 2019, 14(9), 889-906). Compared with noncovalent inhibitors, TCIs can provide increased selectivity, increased potency, and prolonged modulation of target function. Covalent conjugation has enabled inhibition of targets at protein-protein interfaces (PPIs) that were previously thought to be undruggable (Ueda et al. J. Am. Chem. Soc. 2021, 143(12), 4766-4774; Way et al. Curr. Opin. Chem. Biol. 2000, 4(1), 40-46). PPI covalent inhibitors approved by the FDA or in clinical trials have shown great promise in recent years (Deak et al. PNAS USA 2019, 116(18), 8966-8974; Bum-Erdene et al. Cell Chem. Biol. 2019, 26(3), 378-389; He et al. Nat. Commun. 2018, 9, 1-12; Lv et al. Nat. Commun. 2018 9, 5145; Zeng et al. Cell Chem. Biol. 2017, 24(8), 1005-1016; Ishiba et al. Bioorg. Med. Chem. Lett. 2017, 27(11), 2571-2574; Yu et al. Mol. Pharm. 2017, 14(5), 1548-1557; Akcay et al. Nat. Chem. Biol. 2016, 12(11), 931-936; Cardoso et al. Protein Sci. 2012, 21(12), 1885-1896). Currently, suitable chemistry for covalent inhibitor conjugation is dominated by thiol-reactive electrophiles that react with cysteine side chains. Cysteines, however, are rare amino acids and are also poorly represented in PPIs (Way et al. Curr. Opin. Chem. Biol. 2000, 4(1), 40-46; Arkin et al. Chem. Biol. 2014, 21(9), 1102-1114; Cheng et al. J. Hematol. Oncol. 2020, 13(1), 1-14; Abbasov et al. Nat. Chem. 2021, 13(11) 1081-1092). Development of additional chemical warheads for targeting additional residues will further expand the utility of TCIs (Gehringer et al. J. Med. Chem. 2019, 62(12), 5673-5724; Mukherjee et al. Curr. Opin. Chem. Biol. 2018 44, 30-38).

Tyrosines are attractive targets due to their functions in non-covalent interactions including hydrogen bonding, cation-pi or pi-pi interactions, and hydrophobic interactions (Koide et al. ACS Chem. Biol. 2009, 4(5), 325-334; Hett et al. ACS Chem. Biol. 2015, 10(4), 1094-1098; Gambini et al. J. Med. Chem. 2019, 62(11), 5616-5627). While tyrosine is an amino acid of low abundance (3% of protein amino acids) (Bartesaghi et al. Amino Acids 2007, 32(4), 501-515), it is highly enriched at PPIs (Ueda et al. J. Am. Chem. Soc. 2021, 143(12), 4766-4774; Way et al. Curr. Opin. Chem. Biol. 2000, 4(1), 40-46). Specifically, tyrosines are enriched in the histone binding pockets of epigenetic regulator proteins containing bromodomains (BrDs) and chromodomains (ChDs) (Pawson et al. Science 2003, 300(5618) 445-452; Scrutton et al. Biochem. J. 1996, 319, 1-8). Development of adequately selective and potent small-molecule inhibitors for chromodomains, in particular, has shown to be challenging (Santiago et al. J. Comput. Aided. Mol. Des. 2011, 25(12) 1171-1178; Bowkett et al. Chem Med Chem 2018, 13(10), 1051).

The chemistry for targeting tyrosine has largely been limited to sulfonyl fluorides and fluorosulfonates, which react with the phenolic oxygen as the nucleophile (Narayanan et al. Chem. Sci. 2015, 6(5), 2650-2659; Lonsdale et al. Chem. Soc. Rev. 2018, 47(11), 3816-3830; Dong et al. Angew. Chemie Int. Ed. 2014, 53(36), 9430-9448; Zhou et al. Or. Lett. 2018, 20(3), 812-815). However, these warheads are not selective for tyrosine and react with lysine, serine, threonine, and histidine (Gambini et al. J. Med. Chem. 2019, 62(11), 5616-5627; Dong et al. Angew. Chemie Int. Ed. 2014, 53(36), 9430-9448; Martin-Gago et al. Angew. Chemie Int. Ed. 2019, 58(4) 957-966). In addition, they have limited hydrolytic stability, with sulfonyl fluorides displaying particular lability. Additional chemistries have been employed for modifying tyrosine in bioconjugation but have not been employed in ligand-directed chemistry in cells due to high reactivity or poor stability. For example, thiazolinedione (TAD) reagents show rapid reactivity and tyrosine/tryptophan selectivity under mild conditions, but have poor aqueous stability and decompose to the isocyanate, which labels additional residues (e.g., lysine/cysteine) (Decoene et al. ChemRxIV 9 Feb. 2021, DOI 10.26434/chemrxiv.13739320.v1; Ban et al. Bioconjug. Chem. 2013, 24(4), 520-532). Aryl diazonium salts also have been used to conjugate tyrosine, but high reactivity and poor stability limit their use in TCIs (Schlick et al. J. Am. Chem. Soc. 2005, 127(11), 3718-3723; Hooker et al., J. Am. Chem. Soc. 2004, 126(12), 3718-3719; Sengupta et al. Or. Biomol. Chem. 2019, 17(36), 8308-8329).

In 2004, the Francis group reported a three-component Mannich reaction to modify selectively protein tyrosines and tryptophans via the Betti variant of the Mannich reaction (see FIG. 1) (Mannich et al. Arch. Pharm. (Weinheim), 1912, 250(1), 647-667). The Francis reaction is pH-dependent and has slow reaction kinetics. The development of a two-component reaction by the Tanaka lab using a pre-formed, cyclic imine (an iminolactam) improved stability against hydrolysis and increased reactivity to phenols over a broad pH range (see FIG. 1) (Minakawa et al. J. Org. Chem. 2008, 73(21), 8669-8672). But, like the Francis reaction, the Tanaka reaction has slow reaction kinetics and did not show reactivity to tryptophan or pyrrolated lysine.

In view of the foregoing, there remains a need for a method to modify selectively side chains of amino acids, such as tyrosine, tryptophan and pyrrolated lysine, with covalent inhibitors that have improved reaction kinetics, amino acid selectivity, and aqueous stability. It is an object of the present disclosure to provide such a method. This and other objects and advantages, as well as inventive feature, will be apparent from the detailed description provided herein.

### SUMMARY

A method of covalently modifying a tyrosine, tryptophan or pyrrolated lysine in a protein comprising a ligand binding site is provided. The method comprises reacting the side chain of the tyrosine, tryptophan or pyrrol-lysine with a cyclic imine Mannich electrophile appended to a ligand that binds the protein. The cyclic imine can have a structure selected from:

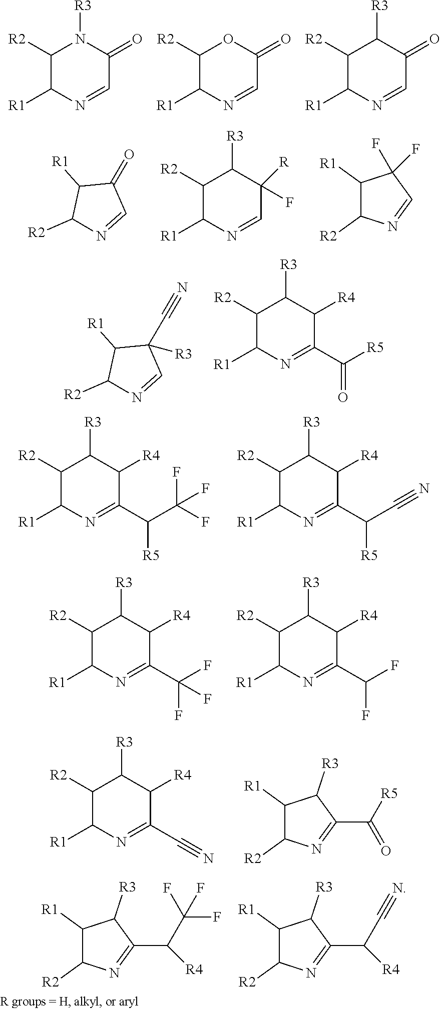

The cyclic imine can be stable in aqueous conditions at a temperature of about 37° C. (e.g., 37° C.) for at least about 12 hours (e.g., 12 hours).

The reacting can be carried out in vitro, e.g., in phosphate-buffered saline, pH 7, at 37° C. The reacting can be carried out in a cell, such as a cell in culture.

The present disclosure provides for a protein comprising a ligand binding site, which comprises one or more tyrosine, tryptophan or pyrrolated lysine residues, the side chains of which have been selectively covalently modified with a cyclic imine. The ligand binding site can further comprise one or more amino acid residues selected from the group consisting of lysine, histidine, serine, threonine, cysteine, glutamic acid, aspartic acid, an N-terminus of the protein, and a C-terminus of the protein, none of which are covalently modified irreversibly with the cyclic imine.

The present disclosure also provides for a protein comprising a ligand binding site, which comprises one or more tyrosine, tryptophan or pyrrolated lysine resides, the side chains of which have been selectively covalently modified by the method disclosed herein.

The present disclosure also provides for a cyclic imine Mannich electrophile appended to a ligand that binds to a target protein, wherein the modified ligand is suitable for reacting the side chain of the tyrosine, tryptophan or pyrrole-lysine of the target protein with the cyclic imine Mannich electrophile appended ligand. It is further provided that the modified ligand is bound by a target protein active site. It is further provided that the modified ligand is bound by the target protein active site with measurable specificity.

The present disclosure also provides for a kit for covalently modifying a tyrosine, tryptophan, or pyrrolated lysine of a target protein, the kit comprising a cyclic imine Mannich electrophile appended to a ligand that binds the protein, suitable for reacting the side chain of the tyrosine, tryptophan or pyrrole-lysine of the target protein with the cyclic imine Mannich electrophile appended ligand.

The present disclosure also provides for a kit for covalently modifying a tyrosine, tryptophan, or pyrrolated lysine of a target protein, the kit comprising a reagent comprising a cyclic imine Mannich electrophile suitable for being appended to a ligand that binds a target protein; a reagent suitable for reacting the side chain of the tyrosine, tryptophan or pyrrole-lysine of a target protein with a cyclic imine Mannich electrophile appended ligand.

### BRIEF DESCRIPTION OF THE DRAWINGS

FIG. 1 compares the provided method with the prior art.

FIG. 2 shows the reaction of cyclic imine Mannich electrophiles (5 mM) with N-acetyl-1-tyrosine methyl amide (100 mM) in phosphate-buffered saline (PBS), pH 7.0, at 37° C. The reaction was monitored using liquid chromatography/mass spectrometry (LC/MS).

FIG. 3 shows second-order rate constants for select cyclic imines with aromatic nucleophiles. Reactions were conducted under pseudo first order conditions with an excess of one reactant. The reduction in the concentration of the limiting reactant A was measured at various time points by HPLC analysis. Rates of imine 1 with N-Ac-Tyr-NMe, indole-3-butyric acid, and 1H-pyrrole-1-propionic acid and of imine 2 with N-Ac-Tyr-NMe were measured.

FIG. 4 shows representative HPLC analysis for second-order rate constants. Reaction of imine 1 (5 mM) with (A) N-Ac-Tyr-NHMe (100 mM) or (B) indole-3-butyrate (100 mM) at indicated time points.

FIG. 5 shows second order rate constant determinations. Reaction of (A) imine 1 (5 mM) with N-Ac-Tyr-NHMe (100 mM) at 37° C. or (B) at room temperature. (C) imine 2 (5 mM) with N-Ac-Tyr-NHMe (100 mM) at 37° C. (D) imine 1 (5 mM) with indole-3-butyrate (100 mM) (E) imine 1 (10 mM) with 1 mM 1H-pyrrole-1-propionate (F) iminium 6 (5 mM) with N-Ac-Tyr-NHMe (100 mM) at 37° C. at pH 7 (G) iminium 6 (5 mM) with N-Ac-Tyr-NHMe (100 mM) at 37° C. at pH 3 at indicated time points.

FIG. 6 shows testing of additional amino acid functional groups for reactivity to imine 1. Cyclic imine 1 was incubated with 100 mM compounds in PBS (pH 7) at 37° C. for 24 hours and then analyzed by LC/MS. (A) Compounds include β-mercaptoethanol, imidazole, isopropanol, n-butylamine, propionic acid, 3-methyl indole, and N-ethyl pyrrole (pyrrole-lysine). UV LC/MS traces for (B) β-mercaptoethanol, (C) 3-methyl indole, and (D) N-ethyl pyrrole. LR-ESI-MS: imine-thiol product m/z calc. for [M+H]⁺ 231.1; found: 231.2. Imine-indole product m/z calc. for [M+H]⁺: 284.2; found: 284.2. Imine-pyrrole product m/z calc. for |M+H|⁺: 248.2; found: 248.2.

FIG. 7 shows the stability of the tyrosine Mannich product. After 24 hours of test reactions in PBS (pH 7) at 37° C., reaction mixture was further incubated with 1% TFA or 1% NaOH or PBS buffer for 96 hours. Mannich product was quantitated by HPLC UV peak integration to evaluate the stability of the product in acidic/basic/neutral conditions.

FIG. 8 shows the reversibility of the thiol-imine reaction. Cyclic imine 1 was reacted with 100 mM cysteine to pre-form the imine-cysteine conjugate, followed up with 1 μM (10 eq) glutathione (GSH), another cysteine containing molecule, or 100 mM (1 eq) acetyl tyrosine methyl amide. Initial imine-cysteine product (imine-thiol product m/z calc. for [M+H]⁺ 231.1; found: 231.2) was reversed by the glutathione or acetyl tyrosine methyl amide to form the new products (imine-glutathione product m/z calc. for [M+H]⁺ 460.2; found: 460.3; imine-tyrosine product m/z calc. for [M+H]⁺ 389.2; found: 389.3). LC/MS analysis showed no remaining cysteine conjugate.

FIG. 9 shows the time-dependent evaluation of thiols on tyrosine labeling with imine 1. Cyclic imine 1 (5 mM) was incubated with 100 mM acetyl tyrosine methyl amide and 5 mM GSH (in the presence of 5 mM TCEP). Reaction yield was quantified based on UV integrations of Mannich product at 4 h, 9 h, 17 h, 24 h, and 32 h. Imine consumption rate was quantified based on the UV integration of Mannich product at 4 h, 9 h, 17 h, 24 h, and 32 h.

FIG. 10 shows the reaction of imine 1 with tyrosine containing peptide angiotensin II. UV trace of reaction mixture of compound 1 (cyclic imine 1) with angiotensin II. Angiotensin II (10 mM) was incubated with imine 1 (50 mM) for 16 hours prior to LC/MS analysis. (Mono cyclic imine-angiotensin II conjugate peak at 2 min): calc. [M+H]⁺: 1199.2, found [M+H]⁺: 1199.3, [M+2H]²⁺: 600.3, [M+3H]³⁺: 400.6. (Di-cyclic imine-angiotensin II conjugate peak at 2.1 min): calc. [M+H]⁺: 1351.2, found [M+H]⁺: 1351.8, [M+2H]²⁺: 676.4, [M+3H]³⁺: 451.0. (Unmodified angiotensin II (8-mer) peak at 2.2 min): m/z calc. [M+H]⁺: 1047.2, found [M+H]⁺: 1047.2, [M+2H]²⁺: 524., [M+3H]³⁺: 350.

FIG. 11 shows the design of covalent inhibitors to the CBX8 chromodomain (ChD). (A) Sequence alignment of the five CBX ChDs in the Polycomb repressive complex I shows a histidine in CBX2 at position 39 that allows the reactivity of the tyrosine in CBX8 to be exploited for improved inhibitor specificity. (B) Crystal structure of peptide ligand UNC3866 bound to CBX8 Chd (PDB code 5EQ0) with Tyr39 adjacent to the diethyl-lysine group. (C) Synthesized derivatives of SW2_110A containing cyclic imine or iminium warheads. Percent covalent conjugation was determined by UV integration on LC/MS after overnight incubation at 10 μM compound and protein at 37° C.

FIG. 12 shows validation of cyclic imine inhibitor P1b stability. Cyclic imine inhibitor P1b was formed by treatment of P1bL with sodium periodate, and the crude sample in PBS was allowed to incubate for 24 hours. The sample was split, and one portion was further reduced with sodium cyanoborohydride. LC/MS: UV traces, low resolution mass spectrum. LR-ESI-MS: m/z calc. for P1bL [M+H]⁺.: 924.6, found: 924.9; m/z calc. for P1b [M+H]⁺: 875.5, found: 875.8; m/z calc. for P1b_NaCNBH3 [M+H]⁺: 878.2, found: 878.2. Stability was similarly assayed with 24-hour incubation in either 100 mM HCl or 100 mM NaOH.

FIG. 13 shows the characterization of ligand directed protein labeling with iminolactone inhibitor P2. (A) The fraction of labeled protein as determined by UV integration on reverse phase HPLC. CBX8 ChD and P2 were incubated at 10 μM each for 16 hours at 37° C. UV absorption measured at 215 nm. (B) Mass spectra of CBX8 ChD treated overnight with inhibitor P2 (top, red) and control CBX8 ChD (bottom, green). Observed masses are shown (expected average mass for CBX8 ChD=8792 Da). The observed mass difference (895 Da) corresponds to the expected mass change of 901 Da. (C) Time dependent fluorescence polarization displacement assays of indicated chromodomains with P2. Assays were conducted using SW2_123 as an FP probe (FIG. S15) at 100 nM. All proteins were used at 2 μM in PBS (pH 7), 0.02% Tween 20. Error bars represent s.d.

FIG. 14 Cellular activity of covalent, cyclic imine inhibitors to CBX8. qRT-PCR analysis of HOXA9 gene expression in THP1 cells after 48 h of compound treatment at the indicated dose. N=3. Error bars represent s.d. Statistical significance determined by Student's T test. *=p<0.05, **=p<0.01, ***=p<0.001, ****=p<0.0001.

FIG. 15 Cellular activity of covalent, cyclic imine inhibitors to CBX8. (A) qRT-PCR analysis of HOXA9 gene expression in THP1 cells after 48 h of compound treatment at the indicated dose. N=3. Error bars represent s.d. Statistical significance calculated using student's t-test. **=p<0.01, ****=p<0.0001. (B) qRT-PCR analysis of HOXA9 gene expression in THP1 cells treated with increasing doses of compound for 48 h. N=3. Error bars represent s.d.

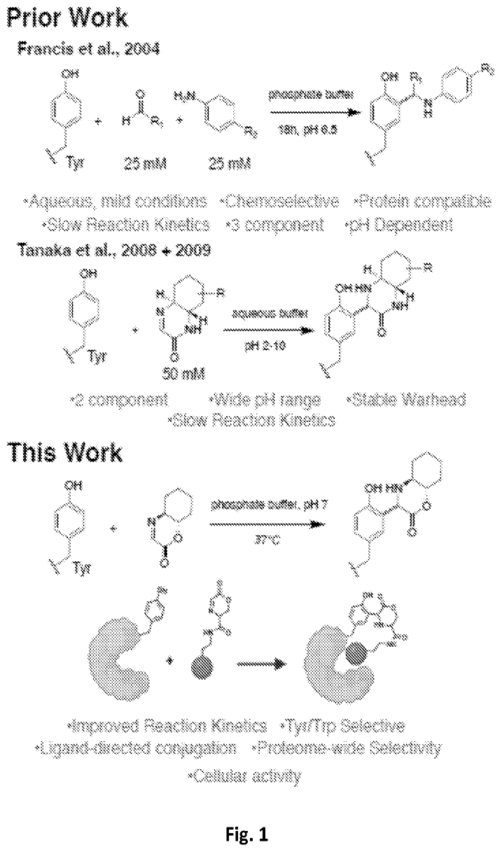

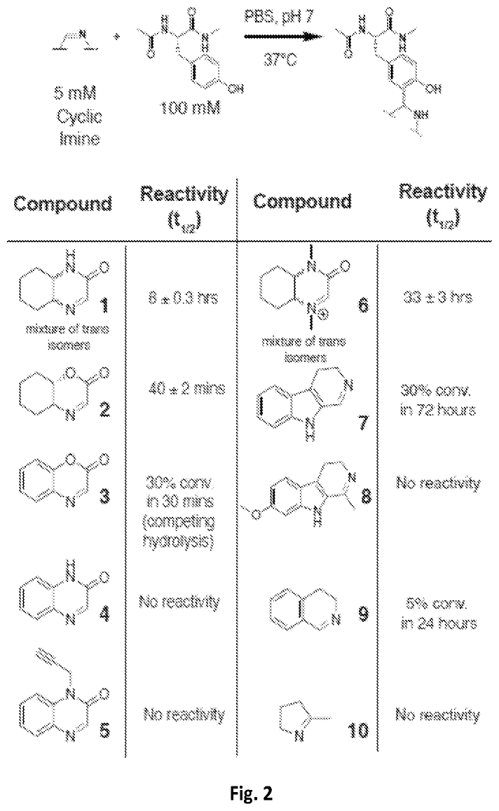

### DETAILED DESCRIPTION

The present disclosure is predicated, at least in part, on the use of two-component Mannich chemistry to modify the side chains of amino acids, such as tyrosines, tryptophans and pyrrolated lysines, in the creation of target covalent inhibitors (TCIs) with in vitro and cellular efficacy. Various cyclic imine derivatives are intrinsically reactive on amino acid mimetics. Thus, inclusion of optimal Mannich electrophiles within or linked to an inhibitor provides a means to create a covalent inhibitor, which can increase inhibitor selectivity and cellular efficacy.

In view of the above, a method of covalently modifying a tyrosine, tryptophan, or pyrrolated lysine in a protein comprising a ligand binding site is provided. The method comprises reacting the side chain of the tyrosine, tryptophan or pyrrole-lysine with a cyclic imine Mannich electrophile appended to a ligand that binds the protein. The cyclic imine can have a structure selected from:

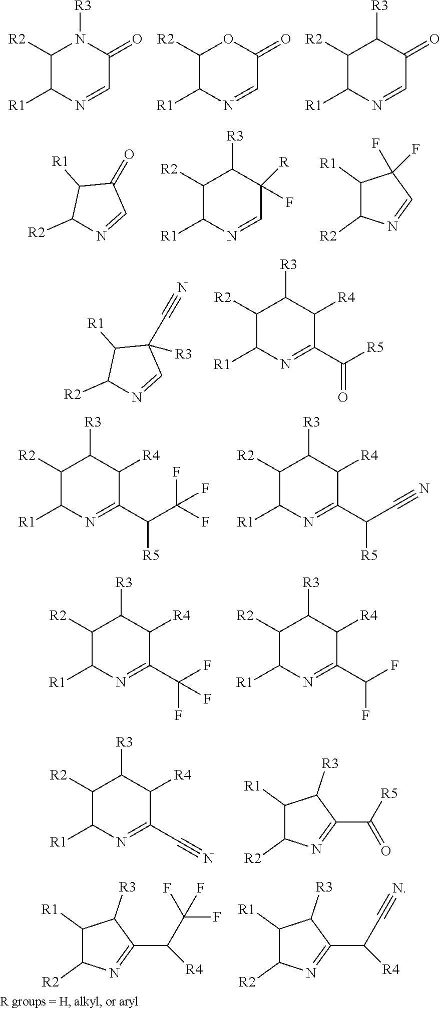

The cyclic imine can be stable in aqueous conditions at a temperature of about 37° C. (e.g., 37° C.) for at least about 12 hours (e.g., 12 hours).

“Alkyl” generally refers to a straight or branched hydrocarbon chain radical consisting solely of carbon and hydrogen atoms, such as having from one to fifteen carbon atoms (e.g., C₁-C₁₅ alkyl). “Alkyl” is intended to include independent recitations of a saturated “alkyl,” unless otherwise stated. An alkyl can comprise one to thirteen carbon atoms (e.g., C₁-C₁₃ alkyl). An alkyl can comprise one to eight carbon atoms (e.g., C₁-C₈ alkyl). An alkyl can comprise one to five carbon atoms (e.g., C₁-C₅ alkyl). An alkyl can comprise one to four carbon atoms (e.g., C₁-C₄ alkyl). An alkyl can comprise one to three carbon atoms (e.g., C₁-C₃ alkyl). An alkyl can comprise one to two carbon atoms (e.g., C₁-C2 alkyl). An alkyl can comprise one carbon atom (e.g., C₁ alkyl). An alkyl can comprise five to fifteen carbon atoms (e.g., C₅-C₁₅ alkyl). An alkyl can comprise five to eight carbon atoms (e.g., C₅-C₈alkyl). An alkyl can comprise two to five carbon atoms (e.g., C₂-C₅ alkyl). An alkyl can comprise three to five carbon atoms (e.g., C₃-C₅ alkyl). In various embodiments, the alkyl group can be selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), and 1-pentyl (n-pentyl). The alkyl is attached to the rest of the molecule by a single bond.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and from five to eighteen carbon atoms, where at least one of the rings in the ring system is fully unsaturated, i.e., it contains a cyclic, delocalized (4n+2) π-electron system in accordance with the Hückel theory. The ring systems from which aryl groups are derived include, but are not limited to, benzene, fluorene, indane, indene, tetralin and naphthalene.

The reacting is carried out under conditions that enable or promote the reaction of the side chain of the tyrosine, tryptophan or pyrrole-lysine with the cyclic imine Mannich electrophile appended to a ligand that binds to the protein. The reacting can be carried out in vitro, e.g., in phosphate-buffered saline, pH 7, at 37° C. The reacting can be carried out in a cell, such as a cell in culture.

The method can be used with any protein comprising a ligand binding site. The ligand can be any ligand with which the protein interacts, whether under normal or abnormal conditions (e.g., diseased conditions). Alternatively, the ligand can be one synthesized to interact with the protein, such as with the ligand binding site of the protein or another site which, upon binding, modulates (e.g., inhibits or promotes) the ability of the ligand binding site to bind a ligand. When the ligand binds to the protein, the reaction of the cyclic imine Mannich electrophile with the side chain of a tyrosine, tryptophan or pyrrole-lysine in or near (e.g., adjacent) the ligand binding site results in a covalent modification, which, depending on the nature of the protein-ligand interaction and any downstream effect(s) of that interaction, can inhibit (i.e., down-regulate or turn “off”) or activate (i.e., up-regulate or turn “on”) the protein and, consequently, any downstream effect(s) of the change in activity of the protein. In various embodiments, the level of inhibition or activation is greater than that realized in the absence of covalent modification.

Thus, in view of the above, also provided is a protein comprising a ligand binding site, which comprises one or more tyrosine, tryptophan or pyrrolated lysine residues, the side chains of which have been selectively covalently modified with a cyclic imine. The ligand binding site can further comprise one or more amino acid residues selected from the group consisting of lysine, histidine, serine, threonine, cysteine, glutamic acid, aspartic acid, an N-terminus of the protein, and a C-terminus of the protein, none of which are covalently modified irreversibly with the cyclic imine.

Thus the present disclosure provides for ligand which are modified with cyclic imine Mannich electrophile, and the modified ligand is bound by the target protein.

It is further provided for in this disclosure, a kit comprising reagents suitable for practicing the method as described above. Such kits can comprise suitably prepared cyclic imine Mannich electrophile reagents for linkage to protein target ligands. Such kit could further comprise suitable supporting reagents, such as buffers, media and the like.

In another embodiment, a kit is provided for where prepared ligand modified with cyclic imine Mannich electrophile is a reagent that is prepared for use in the method as described above.

The kits of the present disclosure can be prepared with complete sets of reagents providing for creation of target protein with one or more tyrosine, tryptophan or pyrrolated lysine residues, the side chains of which have been covalently modified with a cyclic imine.

Such reagents can be provided in bulk, in sets, individually or in other such suitable packaging.

### EXAMPLES

The following examples serve to illustrate the present disclosure. The examples are not intended to limit the scope of the claimed invention in any way.

### Example 1

Reaction of Cyclic Imines with Tyrosine

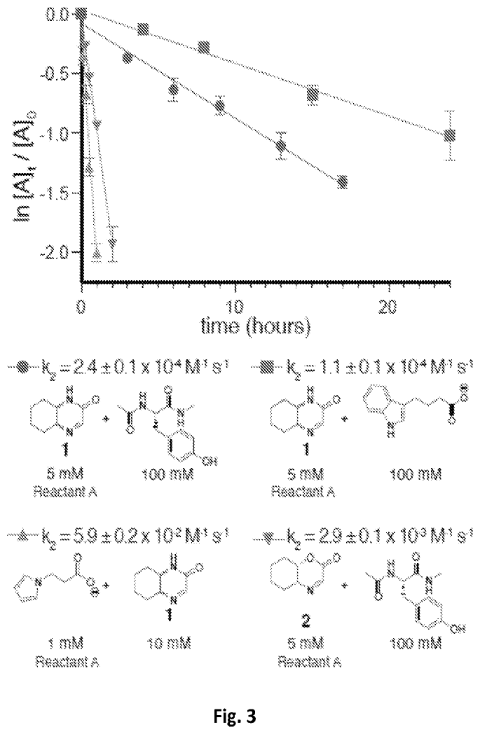

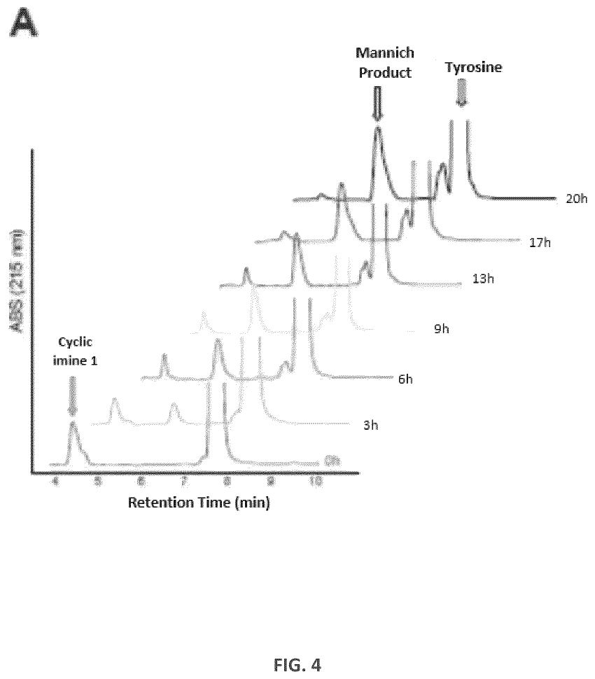

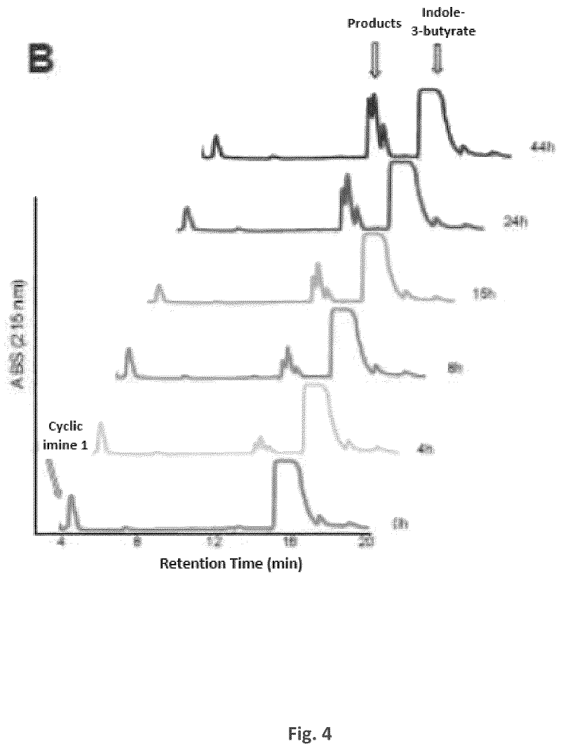

Several cyclic imines (5 mM) were incubated with a simple tyrosine analog N-acetyl-L-tyrosine methylamide (Ac-Tyr-NHMe) in PBS pH 7 at 37° C. (FIGS. 2-3). The reactivity was quantitated by UV integration on HPLC with liquid chromatography/mass spectrometry (LC/MS) confirmation of reaction product identity (FIG. 4). Under these pseudo-first order reaction conditions, reaction half-lives and second-order rate constants were determined.

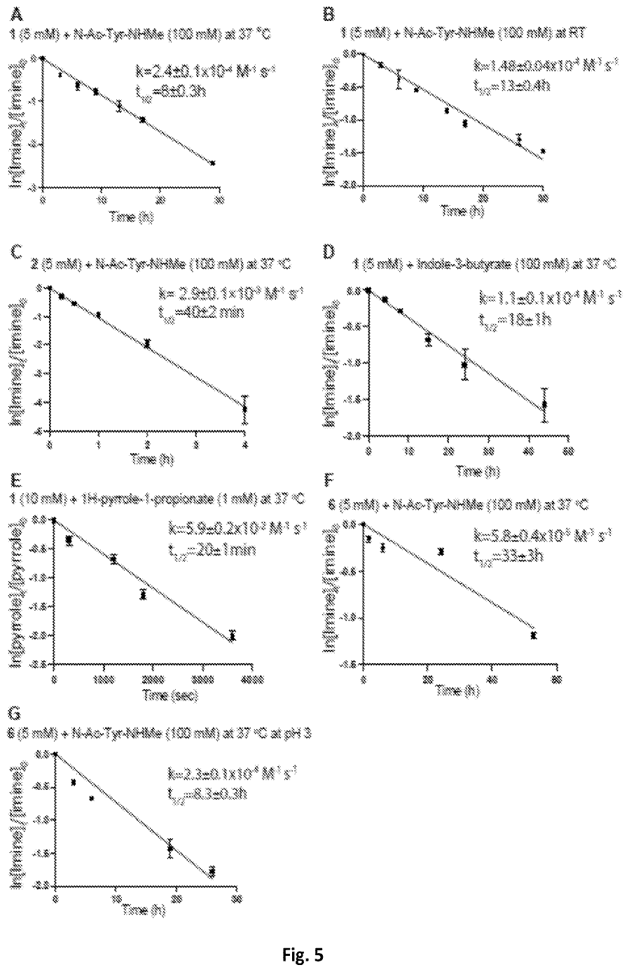

Similar reactivity of the cyclohexyliminolactam 1 to that previously reported by Tanaka et al. was observed using p-cresol (Minakawa et al. J. Org. Chem. 2008, 73(21), 8669-8672). The reaction half-life (t_{1/2}) of imine 1 was 8±0.3 hours under these conditions with a second-order rate constant of 2.4×10⁻⁴ M⁻¹ s⁻¹ at 37° C. (FIG. 3) and 1.5×10⁻⁴ M⁻¹ s⁻¹ at R.T. (FIG. 5). The iminolactone 2 reacted faster with a t_{1/2=40} mins and second-order rate constant of 2.9×10⁻³ M⁻¹ s⁻¹ at 37° C. (FIG. 3). This reaction rate is similar to the intrinsic rates of cysteine to acrylamides (1×10⁻² to 1×10⁻⁴ M⁻¹ s⁻¹), which are commonly used in covalent drugs (Lonsdale et al. J. Chem. Inf Model. 2017, 57(12), 3124-3137). The ~10-fold increase in rate constant of 2 relative to 1 indicates the tunability of imine reactivity with adjacent electron withdrawing groups. Further increasing electron withdrawing properties using aromatic ester imine 3 did increase the rate of Mannich addition, but hydrolysis of the phenol ester was a competing reaction. While stable in water, we observed 70% hydrolysis of 3 in PBS, pH 7, within an hour. Compound 4, with the less reactive amide, (drawn as the analogous tautomer of 2-hydroxyquinoxaline in FIG. 2) was unreactive, which was anticipated due to aromaticity. It was hypothesized that N-alkylation with a propargyl group would increase the tautomeric preference towards the reactive imine; however, compound 5 was also unreactive. Since protonation of the imine can be rate-limiting in the Mannich reaction (Cummings et al. J. Org. Chem. 1960, 25(3), 419-423), it was reasoned that an iminium may lead to enhanced reactivity, similar to recent work with a proline-selective Petasis reaction (Sim et al. Chem. Sci. 2020, 11(1), 53-61). Iminium 6, however, was less reactive than other imines (t₁₋₂~33 hours, FIG. 5), presumably due to equilibrium favoring either the hydrate or the open, aldehyde forms (Eldin et al. 1995, 117(17), 4851-4857). Commercially available di-hydro-beta-carbolines showed either very weak (compound 7) or no reactivity (compound 8). Other imines showed limited (compound 9) to no (compound 10) reactivity to tyrosine, as summarized in FIG. 2.

### Example 2

### Reactivity of Cyclic Imines to Additional Amino Acid Mimetics

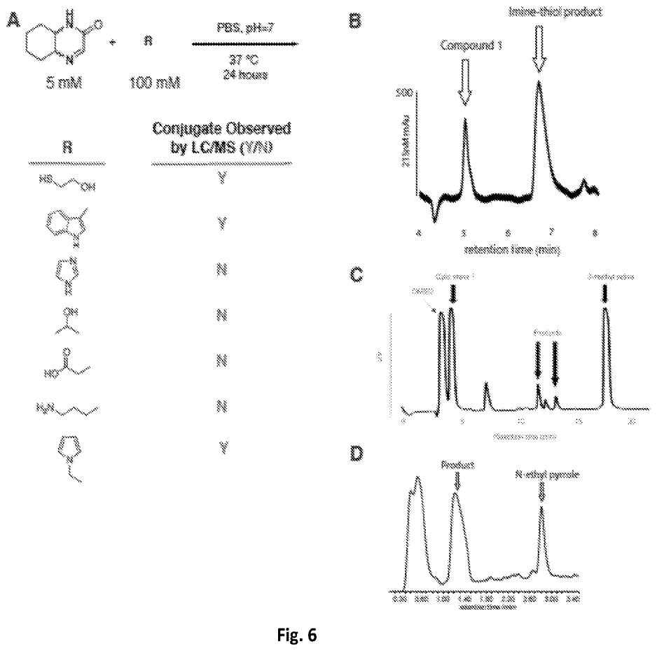

To assess the selectivity to other amino acids, we tested compounds mimicking amino acid side chain functional groups, including β-mercaptoethanol (cysteine), imidazole (histidine), isopropanol (serine/threonine), n-butylamine (lysine), propionic acid (aspartic acid/glutamic acid), 3-methyl indole (tryptophan), and N-ethyl pyrrole (pyrrole-lysine). Stable conjugates to 1 for β-mercaptoethanol, 3-methyl indole, and N-ethyl pyrrole were observed by LC/MS analysis (FIG. 6). Conjugation to thiols was expected based on previous reports (Minakawa et al. J. Org. Chem. 2008, 73(21), 8669-8672; Cavaca et al. Mol. 2022, 27(5), 1673). While partially stable under LC/MS conditions, the N,S-thioaminal product was not stable under stronger acidic conditions (0.1 μM HCl) and was readily reversible (Cavaca et al. Mol. 2022, 27(5), 1673).

The tryptophan mimic, 3-methyl indole, reacts with 1 to form multiple products, likely a mixture of N-substitution and C2/C3- Mannich- or Pictet Spangler-type additions (McFarland et al. J. Am. Chem. Soc. 2008, 130(24), 7639-7644; Antos et al. J. Am. Chem. Soc. 2004, 126(33), 10256-10257; Larda et al. Bioconjug. Chem. 2015, 26(12), 2376-2383) (FIG. 6). The poor aqueous solubility of 3-methyl indole made it challenging to obtain a rate constant and may explain conflicting results from the Tanaka group that did not report reactivity with imine 1 (Minakawa et al. J. Org. Chem. 2008, 73(21), 8669-8672). Using indole-3-butyrate as an alternative tryptophan mimic with enhanced solubility, we determined the rate constant (k2=1.1×10⁻⁴ M⁻¹ s⁻¹ at 37° C.) (FIGS. 3-5), which was only 2-fold lower than the rate constant to tyrosine.

We also tested 1 with N-ethyl pyrrole as a mimic of pyrrolated lysine, a lesser-known post-translational modification (Miyashita et al. Sci. Rep. 2014, 4, 5343; Chikazawa et al. J. Biol. Chem. 2020, 295(22), 7697-7709). Pyrroles are particularly electron-rich aromatic groups that react rapidly in Mannich-type reactions. Previous work with N-pyrrolyl alanine derivatives has shown fast and selective Pictet-Spengler reactions with aldehyde-containing biomolecules (Pomplun et al. Angew. Chemie Int. Ed. 2019, 58(11), 3542-3547).⁵⁰ Under conditions of 10 mM cyclic imine 1 with 1 mM 1H-pyrrole-1-propionate (as a soluble pyrrole-Lys mimic), we determined a second order rate constant of 5.9×10⁻² M⁻¹ s⁻¹ (FIGS. 3 and 5). This ~200-fold increase relative to Tyr suggests cyclic imines may be effective reactive groups for proteomic profiling of lysine pyrrolation.

### Example 3

Stability and Reversibility of Cyclic Imines with Tyrosine and Cysteine

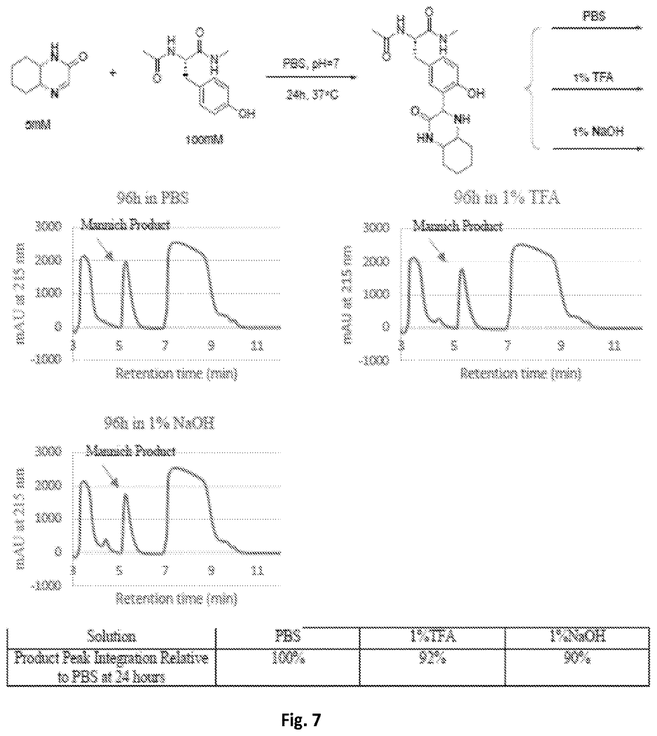

To validate the stability of cyclic imines as covalent warheads, imines 1-4 were evaluated by LC/MS after 96-hours incubation in PBS, 0.1M HCl, and 0.1M NaOH (Table 1). Compounds 1 and 4 were stable under all conditions. Both ester-containing compounds 2 and 3 hydrolyzed under basic conditions. Aromatic ester imine 3 was unstable in PBS, with a mixture of hydrolysis and Mannich reaction with the hydrolysis product 2-amino phenol. The stability of the product of imine 1 with Ac-Tyr-NHMe in 0.13 μM (1%) TFA, 0.53 μM (1%) NaOH, or PBS buffer for 96 hours was evaluated, and only marginal decomposition of the Tyr Mannich product was observed (FIG. 7).

TABLE 1

Compounds

| | 1 | 2 | 3 | 4 |
|---|---|---|---|---|
| H₂O | + | + | + | + |
| PBS | + | + | − | + |
| 100 mM HCl | + | + | + | + |
| 100 mM NaOH | + | − | − | + |

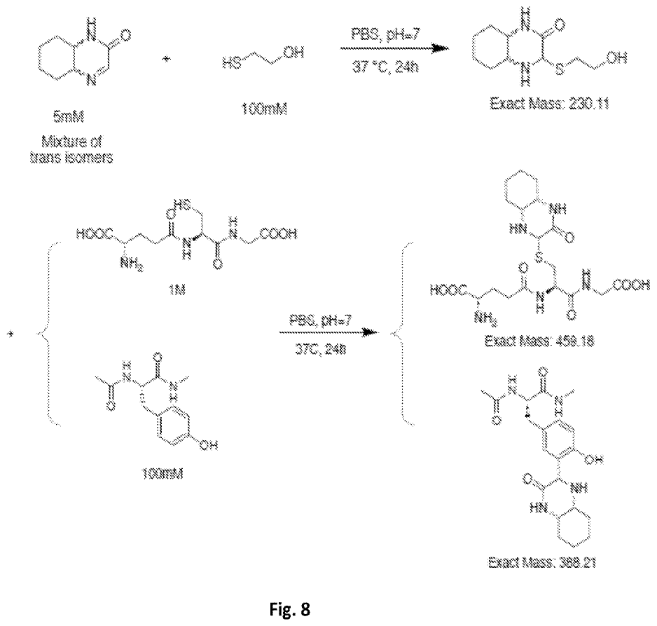

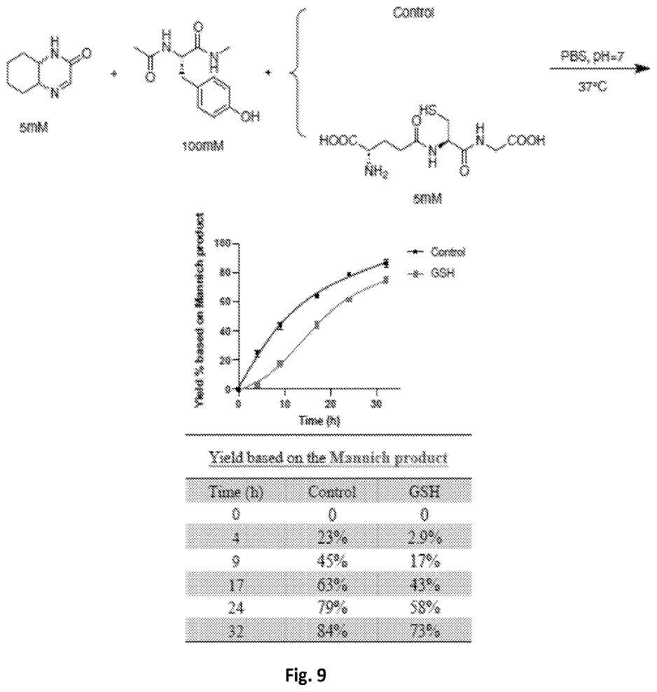

Thiol addition to the imine is reversible, yet this reaction may limit the desired reactivity of imines to tyrosines in cellular contexts where glutathione concentrations can be 1-10 mM (Meister et al. J. Biol. Chem. 1988, 263(33), 17205-17208; Forman et al. Mol. Aspects Med. 2009, 30(1-2), 1). To evaluate this reversibility, the pre-formed thioaminal product of 1 with N-acetyl-cysteine methyl amide was treated with either glutathione (1 M) or Ac-Tyr-NHMe (100 mM). Both glutathione and Ac-Tyr-NHMe outcompeted the cysteine product after 24 hours (FIG. 8). To evaluate potential effects of cellular thiols on tyrosine labeling rate, the cyclic imine-Tyr reaction (at 5 mM and 100 mM, respectively) was evaluated over time in the presence of cysteine (5 mM) (FIG. 9). The presence of cysteine slightly slowed the reaction rate of tyrosine-imine compared to the control.

### Example 4

Covalent Labeling of Cyclic Imine with Tyrosine-Containing Peptide (Angiotensin II) and Protein (CBX8 Chromo-Domain)

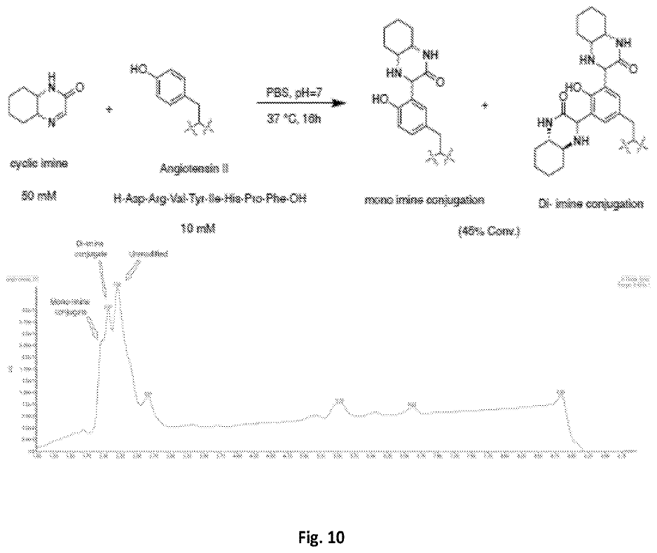

To confirm reactivity and selectivity with a peptide context, imine 1 (50 mM) was incubated with the tyrosine-containing 8-mer peptide angiotensin II (DRVYIHPF) (10 mM) (FIG. 10). The combined yield of mono-labeled and di-labeled angiotensin II was ~45% as quantified by LC/MS. Cyclic imine 1 (50 mM) was also incubated with a small protein (recombinant CBX8 chromodomain (ChD), which contains multiple tyrosines and tryptophans; 10 μM in PBS buffer (pH=7; 0.02% Tween 20)) at room temperature overnight. With overnight incubation, two to three additions of the imine were observed by mass spectrometry.

### Example 5

### Design and Synthesis of Cyclic Imine-Containing CBX8 ChD Inhibitors

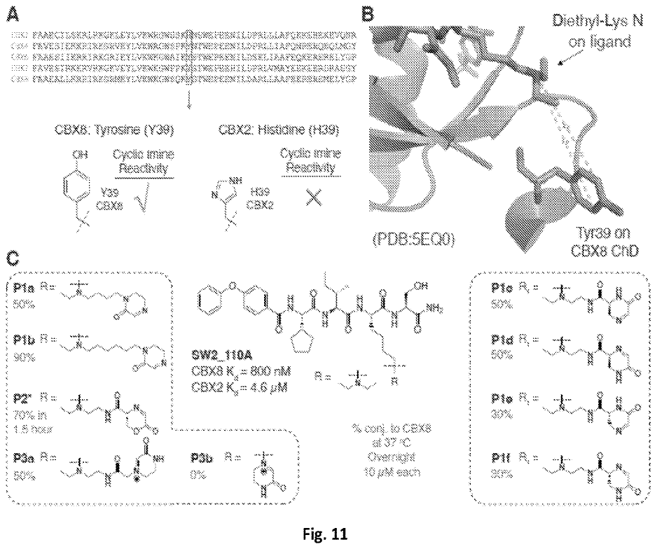

SW2_110A is a selective CBX8 ChD inhibitor with an 800 nM K_{d} (FIG. 11C) (Wang et al. ACS Chem. Biol. 2020, 15(1), 112-131; Denton et al. SLAS Discov. 2018, 23(5), 417-428). Although SW2_110A demonstrates high selectivity for CBX8 over CBX4, CBX6, and CBX7, it is only 6-fold selective over CBX2. Amino acid sequence alignment and an X-ray crystal structure of a similar ligand (UNC3866) bound to CBX8 (FIG. 11B) (Stuckey et al. Nat. Chem. Biol. 2016, 12(3), 180-187) were used to identify a tyrosine (Y39) residue adjacent the diethyl lysine binding site that is a histidine in CBX2. One of the ethyl groups of the alkylated lysine side chain of the ligand is solvent exposed and points in the direction of Y39 on CBX8, which is ~8-9 Å from the lysine nitrogen (FIG. 11B). Thus, several potential covalent ligands were synthesized by linking warheads off the ethyl group of SW2_110A. (FIG. 11C).

The analogs were synthesized using standard, solid phase peptide synthesis of SW2_110A with a nosyl-protected lysine (Schemes S7-S13), which was further elaborated on-resin by alkylation with an alkylhalide or Fukuyama-Mitsunobu chemistry (Kan et al. Chem. Commun. 2004, 353-359). Due to concerns about imine stability throughout synthesis, the warhead was prepared as a primary amine suitably placed adjacent to a serine amide or ester as a latent electrophile. After purification, the compounds were treated in situ with sodium periodate to oxidize the 1,2-amino alcohol of the serine to produce an alpha-aldehyde amide/ester, which then spontaneously cyclized to give the desired imines.

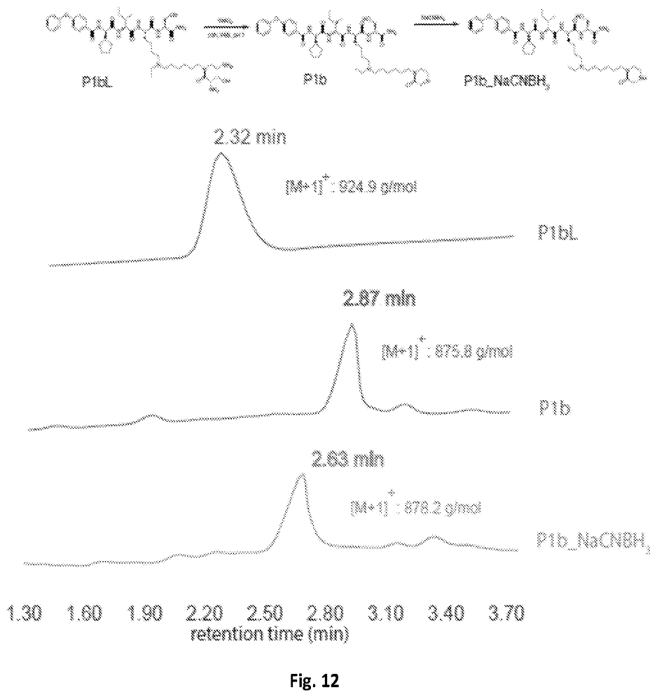

To rule out concerns that the phenoxyphenyl moiety of SW2_110A may self-react with the warhead, a 24-hour incubation of P1b in PBS followed by treatment with sodium cyanoborohydride (10 eq.) was performed. Mass spectrometry indicated full conversion of imine to amine (FIG. 12). Similarly, the longer-term stability of P1b in acidic (100 mM HCl), basic (100 mM NaOH), and neutral PBS buffers was tested, and no significant degradation after 96 h at room temperature was observed.

### Example 6

### Covalent Labeling of Cyclic Imine Containing Ligands to CBX8 ChD

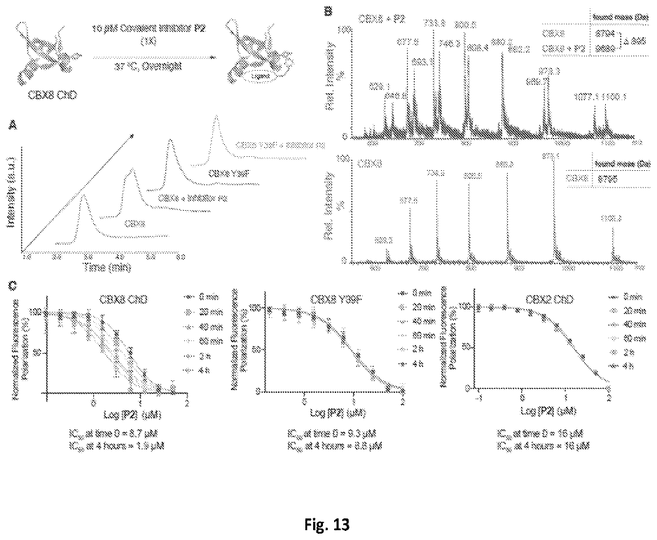

To evaluate the reaction with CBX8 ChD, all cyclic imine ligands were tested with purified CBX8 ChD. To saturate the binding of inhibitor to protein, 10 μM of the covalent inhibitors were incubated with 10 μM CBX8 ChD (~10-fold over the K_{d}) in PBS buffer at 37° C. for 12 hours. The labeling of inhibitors to CBX8 ChD was quantified by LC/MS (FIGS. 11C, 13A, and 13B). Among the cyclic amide imine series (Pt's), compound Pla, with a 6-carbon linker, gave the best yield for labeling the CBX8 ChD (~90%). This compound was also tested in the presence of 10 mM GSH, and no effect on yield was observed. Due to the enhanced reactivity of iminolactone (warhead 2), CBX8 ChD labeling using ester imine inhibitor P2 was considerably faster, and around 70% protein labeling was achieved in 1.5 hour (FIGS. 13A and 13B). We additionally prepared two iminium warhead compounds, P3a and P3b. Despite the low intrinsic reactivity observed with model iminium 3 and Tyr (Table 1), inhibitor P3a gave a labeling yield (50%) that was comparable to the more reactive iminolactams. This may suggest assistance in the reaction from the local environment on the protein.

### Example 7

### Time-Dependent Fluorescence Displacement Assay

A fluorescence polarization (FP) displacement assay was used to evaluate the covalent binding of cyclic imine warhead containing inhibitors. Without additional incubation time, the non-covalent inhibitor SW2_110A and optimal covalent inhibitors P1b (iminolactam) and P2 (iminolactone) had comparable IC₅₀ values, which suggests that the structural modification of the warhead does not significantly change the affinity. With further incubation, P1b and P2 displayed a time-dependent increase of potency (decrease of IC₅₀), while non-covalent inhibitor SW2_110A did not (FIG. 13C). To validate protein stability over the incubation time of the assay, the K_{d} of the FP probe (SW2_123) was measured at time 0 and 20 hours and determined to be unchanged, indicating good stability.

### Example 8

### Validation of CBX8 Labeling at Y39

To validate labeling of the targeted tyrosine residue (Y39), this amino acid was mutated to phenylalanine, which is structurally similar but lacks reactivity to the cyclic imine. The fluorescence displacement assay indicated inhibitors bind to wild-type or mutant CBX8 (Y39F) with comparable IC₅₀ values (9.3 μM versus 8.7 μM for P2, 7.4 μM versus 7.3 μM for P1b, respectively) (FIG. 13C); however, both the time-dependent FP assay (FIG. 13C) and MS protein labeling experiments (FIG. 11A) indicate that covalent inhibitors fail to label the mutant CBX8 Y39F. Similarly, for CBX2 ChD, no change in apparent affinity was observed over time in the FP assay (FIG. 13C), and no labeled protein was detected by MS. Together these results support the ligand-directed labeling of Y39 on CBX8.

### Example 9

### Proteomic Selectivity of Cyclic Imine Inhibitor

To demonstrate the covalent labeling of endogenous CBX8, in-gel fluorescence labeling was performed. After incubation of the inhibitor P2 at 1 μM in HEK293T cell lysates for 1 h at 37° C., click chemistry was conducted to conjugate TAMRA-azide to the C-terminal alkyne of P2 prior to SDS-PAGE. Gel analysis showed a prominent fluorescent band, which is approximately the molecular weight of CBX8. With co-incubation of excess non-covalent inhibitor SW2_110A (~20 μM, 20 eq), protein labeling was reduced. The dose-dependency of labeling of CBX8 with the covalent inhibitor from nM to 1000 nM inhibitor concentration was additionally demonstrated.

To corroborate that imine inhibitors can covalently capture the endogenous, full-length CBX8, pulldown assays were also performed making use of propargyl amide modified inhibitors and click chemistry to biotin-azide. Besides P2, we prepared a propargyl amide derivative of 6-carbon linker iminolactam inhibitor, P1bP (Scheme S5). Covalent inhibitors P2, P1bP, and a non-covalent inhibitor SW2_110A were incubated with 293T cell lysates at 1 μM for protein labeling. For competition, an excess (10-100 eq) of the non-covalent inhibitor was added. Labeled lysates were then clicked to a biotin-azide for affinity purification. Under non-denaturing washing buffer conditions, both covalent and non-covalent inhibitors enriched CBX8 from lysates. Using more stringent washing conditions, only covalent inhibitor P2 enriched CBX8 from lysates. To remove non-specific background from highly expressed CBX7, and the abundant nuclear protein BRG1, fully denaturing washing conditions of 0.2-1% SDS in PBS or 6M urea were used (Weerapana et al. Nat. Protoc. 2007, 2, 1414-1425; Kurzban et al. J. Biol. Chem. 1991, 266(22), 14470-14477). Under these conditions, CBX8 was still captured by the covalent inhibitor, while other CBXs, known CBX8 interacting protein RING1B, or non-specific protein BRG1 were not. To further demonstrate ligand-dependent covalent labeling, an excess of the non-covalent inhibitor was doped in for competition. With 100 equivalents of competing ligand added, covalent labeling of CBX8 by either compound P2 or P1bP was reduced, as quantitated by western blot.

To evaluate and quantitate covalent ligand activity in whole cell proteomes, mass spectrometry analysis was performed after compound pulldown (chemoIP-MS). CBX8 was robustly enriched using the biotinylated, covalent inhibitor P2-B. Of the nine proteins significantly enriched with P2-B (log 2(P2-B/DMSO)>3, P<0.001), only CBX8 enrichment was significantly reduced with competing non-reactive ligand SW2_110A, indicating ligand-dependent, covalent labeling of endogenous CBX8 from lysates. Results were similar using iminolactam inhibitor P1bP.

### Example 10

### Cellular Activity of Covalent Inhibitors in MLL-AF9 Transformed Leukemia Cell Line

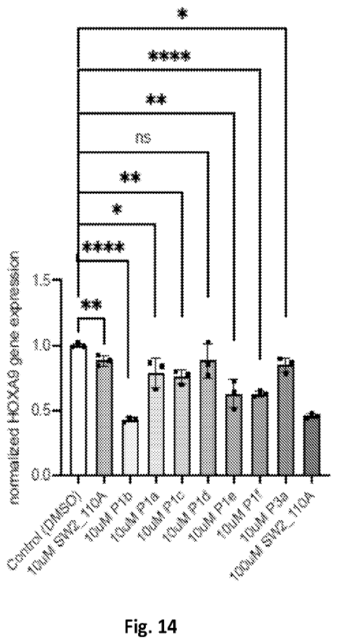

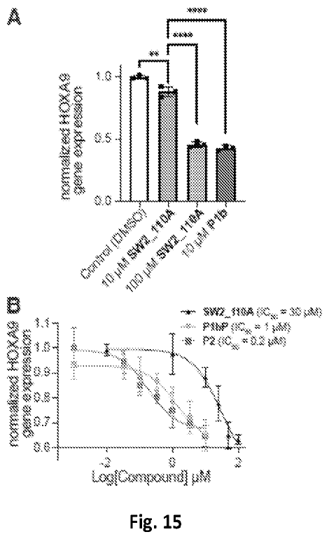

Imine inhibitors were assessed for improved cellular activity using a disease-relevant cell model. We previously reported that the non-covalent inhibitor SW2_110A inhibits CBX8 binding to chromatin and reduces CBX8-mediated gene expression in MLL-AF9 transformed leukemia (Wang et al., ACS Chem. Biol. 2020, 15(1), 112-131). Using this same model, we compared the covalent inhibitors with non-covalent inhibitor SW2_110A in THP1 cell lines. Initially, P1 series inhibitors and P3a reduced expression to a greater extent than the non-covalent inhibitor (FIG. 14). Consistent with it having the greatest in vitro reactivity among those tested, inhibitor P1b was the most effective at reducing HOXA9 gene expression where 10 μM P1b treatment reduced gene expression to a similar extent as 100 μM SW2_110A (FIG. 15A). The two optimal inhibitors P1bP and P2 were subsequently tested for dose-dependency in this cellular assay. Dose curves indicate that these covalent inhibitors have 30 to 100-fold improvement of cellular activity compared to the non-covalent inhibitor (FIG. 15B).

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The disclosure illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “substantially” can allow for a degree of variability in a value or range, for example, within 90%, within 95%, or within 99% of a stated value or of a stated limit of a range.

The terms and expressions, which have been employed, are used as terms of description and not of limitation. In this regard, where certain terms are defined under “Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Definitions,” are used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered within the scope of the invention as claimed herein.

1. A method of covalently modifying a tyrosine, tryptophan, or pyrrolated lysine in a protein comprising a ligand binding site, which method comprises reacting the side chain of the tyrosine, tryptophan or pyrrole-lysine with a cyclic imine Mannich electrophile appended to a ligand that binds the protein.

2. The method of claim 1, wherein the cyclic imine has a structure selected from: 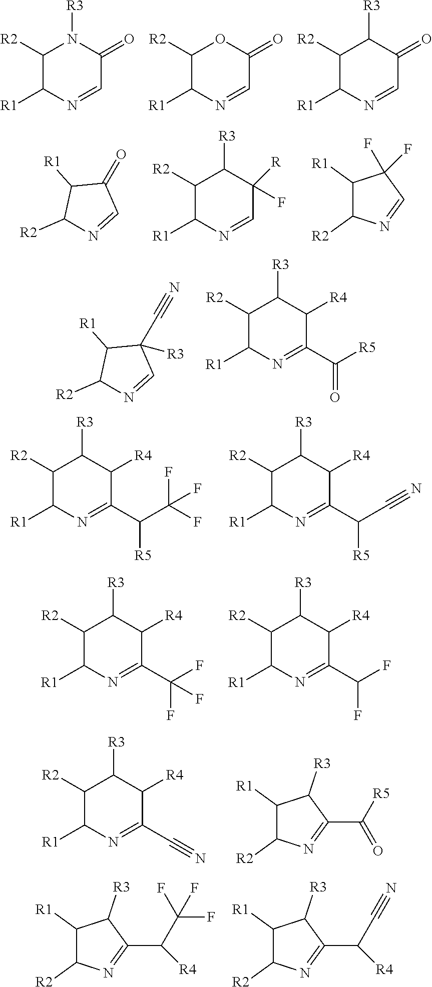
where R1, R2, R3, R4, and R5 is H, aklyl, or aryl.

3. The method of claim 2, wherein the cyclic imine is stable in aqueous conditions at a temperature of about 37° C. for at least about 12 hours.

4. The method of claim 1 wherein the reacting is carried out in phosphate-buffered saline, pH 7, at 37° C.

5. The method of claim 1 wherein the reacting is carried out in cell culture.

6. A protein comprising a ligand binding site, which comprises one or more tyrosine, tryptophan or pyrrolated lysine residues, the side chains of which have been selectively covalently modified with a cyclic imine.

7. The protein of claim 6, wherein the ligand binding site further comprises one or more amino acid residues selected from the group consisting of lysine, histidine, serine, threonine, cysteine, glutamic acid, aspartic acid, an N-terminus of the protein, and a C-terminus of the protein, none of which are covalently modified irreversibly with the cyclic imine.

8. A protein comprising a ligand binding site, which comprises one or more tyrosine, tryptophan or pyrrolated lysine resides, the side chains of which have been selectively covalently modified by the method of claim 1.

9. A kit for covalently modifying a tyrosine, tryptophan, or pyrrolated lysine of a target protein, the kit comprising a cyclic imine Mannich electrophile appended to a ligand that binds the protein, suitable for reacting the side chain of the tyrosine, tryptophan or pyrrole-lysine of the target protein with the cyclic imine Mannich electrophile appended ligand.

10. The kit of claim 9 further comprising phosphate-buffered saline.

11. The kit of claim 9 further comprising cell culture media.

12. A kit for covalently modifying a tyrosine, tryptophan, or pyrrolated lysine of a target protein, the kit comprising a reagent comprising a cyclic imine Mannich electrophile suitable for being appended to a ligand that binds a target protein; a reagent suitable for reacting the side chain of the tyrosine, tryptophan or pyrrole-lysine of a target protein with a cyclic imine Mannich electrophile appended ligand.

13. The kit of claim 12 further comprising a suitable buffered solution.

14. The kit of claim 12 wherein the cyclic imine is selected from the group consisting of: 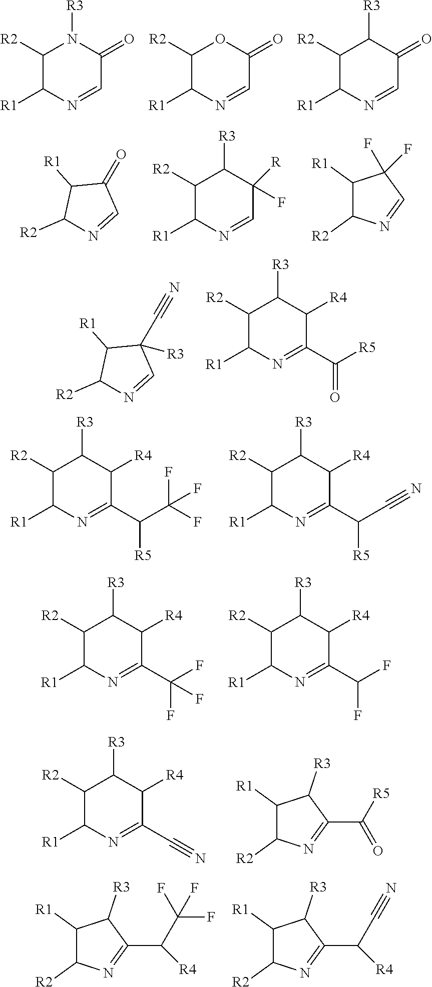
where R1, R2, R3, R4, and R5 is H, aklyl, or aryl.


SEQUENCE LISTING (6 sequences)

SEQ ID NO: 1 - protein, 59 aa, unidentified
FAAECILSKRLRKGKLEYLVKWRGWSSKHNSWEPEENILDPRLLLAFQKKEHEKEVQNR

SEQ ID NO: 2 - protein, 59 aa, unidentified
FAVESIEKKRIRKGRVEYLVKWRGWSPKYNTWEPEENILDPRLLIAFQNRERQEQLMGY

SEQ ID NO: 3 - protein, 59 aa, unidentified
FAAESIIKRRIRKGRIEYLVKWKGWAIKYSTWEPEENILDSRLIAAFEQKERERELYGP

SEQ ID NO: 4 - protein, 59 aa, unidentified
FAVESIRKKRVRKGKVEYLVKWKGWPPKYSTWEPEEHILDPRLVMAYEEKEERDRASGY

SEQ ID NO: 5 - protein, 59 aa, unidentified
FAAEALLKRRIRKGRMEYLVKWKGWSQKYSTWEPEENILDARLLAAFEEREREMELYGP

SEQ ID NO: 6 - protein, 8 aa, unidentified
DRVYIHPF